(19) United States
(12) Plant Patent Application Publication
(10) Pub. No.: US 2003/0126662 P1
(43) Pub. Date: Jul. 3, 2003

(54) Mahaleb rootstock named 'UCMH 55'

(76) Inventor: Stephen M. Southwick, Davis, CA (US)

Correspondence Address: BURNS DOANE SWECKER & MATHIS L L P, POST OFFICE BOX 1404, ALEXANDRIA, VA, 22313-1404, US

(21) Appl. No.: 10/028,771

(22) Filed: Dec. 28, 2001

(51) Int. Cl. A01H 5/00

(52) U.S. Cl. PLT/317

(57) ABSTRACT

A new and distinct cultivar of Prunus mahaleb is provided. The new cultivar is particularly well suited for serving as an understock during cherry production. A number of advantages are provided when compared to the standard Mahaleb rootstock. The cultivar is readily amenable to vegetative propagation (e.g., by the use of softwood cuttings), and exhibits improved resistance to Phytophthora spp. When used with a ‘Bing’ cherry scion, increased yields have been observed. The new cultivar when grown without use as an understock forms a tree that is intermediate in size and forms more fine wood when compared to the ‘UCMH 56’ and ‘UCMH 59’ cultivars that were products of the same research program.

### SUMAMRY OF THE INVENTION

Mahaleb rootstocks (i.e., Prunus mahaleb rootstocks) are widely used during both sweet and sour cherry production throughout the world. It has been the common practice to form such rootstock plants from seed following the random outcrossing of parent plants. Accordingly, cherry production encountered when using such plants as an understock has tended to be somewhat variable due to differences in the genotype of the understock. Such variation often has led to reduced field performance on some cherry trees on a random and unpredictable basis. Mahaleb rootstocks in the past have generally been found to be incapable of vegetative propagation on a reliable basis, such as through the use of softwood and hardwood cuttings. Also, such rootstocks in the past have been susceptible to root and crown fungal diseases generally known as Phytophthora spp.

### SUMMARY OF THE NEW CULTIVAR

It was an object of my research to provide Prunus mahaleb rootstocks that possess characteristics that overcome shortcomings of the Mahaleb rootstock presently being used during cherry production. More specifically, it was my goal to provide cherry rootstocks that could be vegetatively propagated in an expeditious and reliable manner so that cherry growers can eliminate crop variation that can be traced to lack of uniformity in the rootstock. Also, it was a goal of my research to provide new Mahaleb rootstocks that inherently display needed resistance to disease and thereby make possible a satisfactory cherry crop on a more consistent basis combined with a reduction in the need to replant because of tree loss that is traceable to disease.

The original tree of the new Prunus mahaleb cultivar of the present invention was discovered through detailed evaluation and selection while growing in a cultivated area at the Experimental Orchards at the University of California located at Davis, Calif., U.S.A. The exact parentage of the new cultivar is unknown. The seeds used to form the planting where the discovery took place came from a random collection of wild Prunus mahaleb germplasm that had been collected from around the world. The large number of seedlings present in the planting were carefully studied and evaluated and a single plant possessing the combination of characteristics of the new cultivar of the present invention was selected and was preserved. Had this plant not been discovered and preserved, it would have been lost to mankind.

Other Prunus mahaleb cultivars resulting from the same research are ‘UCMH 56’ (U.S. Plant Pat. No. ______, filed concurrently herewith), and ‘UCMH 59’ (U.S. Plant Pat. No. ______, filed concurrently herewith).

It was found that the new Prunus mahaleb cultivar of the present invention exhibits the following combination of characteristics:

(a) readily is amenable to vegetative propagation,

(b) performs well as an understock for cherry production,

(c) forms a tree that is intermediate in size and forms more fine wood when compared to the ‘UCMH 56’ and ‘UCMH 59’ cultivars, and

(d) displays improved resistance to Phytophthora spp.

In view of the above combination of characteristics, the new cultivar of the present invention well meets the needs of cherry producers for use as an improved rootstock. Cherry scion characteristics are no longer influenced by variation in the Mahaleb rootstock resulting from the random outcrossing of parental plants. Also, the disease resistance made possible by the new cultivar is a major advantage for cherry producers.

The new cultivar of the present invention has been repeatedly reproduced through the use of softwood and hardwood cuttings at Davis, Calif., U.S.A. Such propagation has confirmed that the characteristics of the new cultivar are stable and are firmly fixed and are transmitted to subsequent generations on a reliable basis.

The new cultivar of the present invention initially was designated ‘UC MAHALEB 155-1’, and subsequently has been named ‘UCMH 55’.

### BRIEF DESRIPTION OF THE PHOOTOGRAPHS

The accompanying photographs show specimens of the plant and plant parts, and also provide DNA information concerning the new cultivar of the present invention. Color is shown as nearly true as is possible to make the same in color illustrations of this character. The trees of the new cultivar were grown at the Experimental Orchards of the University of California located at Davis, Calif., U.S.A.

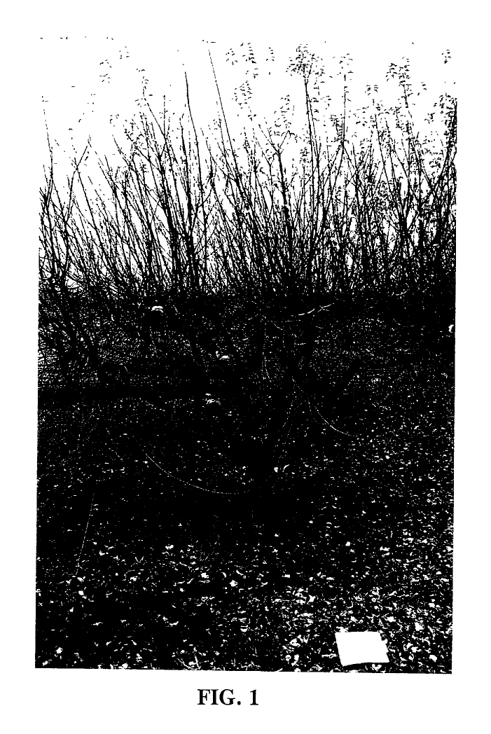

FIG. 1 shows a tree of approximately 5 to 7 years of age during December. The tree is a mother plant that was being used to make propagules for additional testing and evaluation. Most of the leaves had dropped by the end of the preceding October.

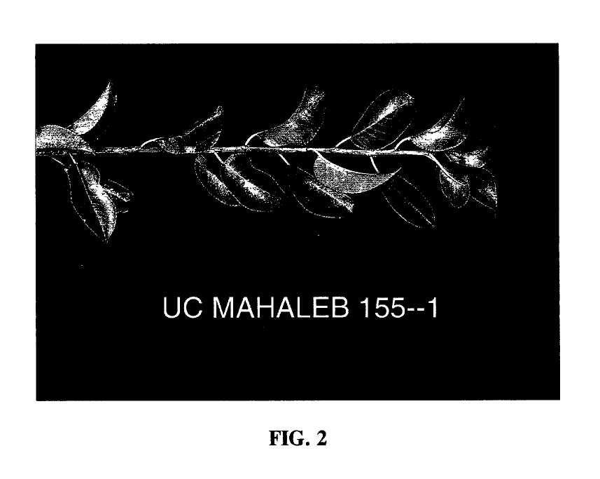

FIG. 2 shows a specimen of a current season's shoot with leaves collected during mid-October. Such shoot was suitable for use to make a hardwood cutting.

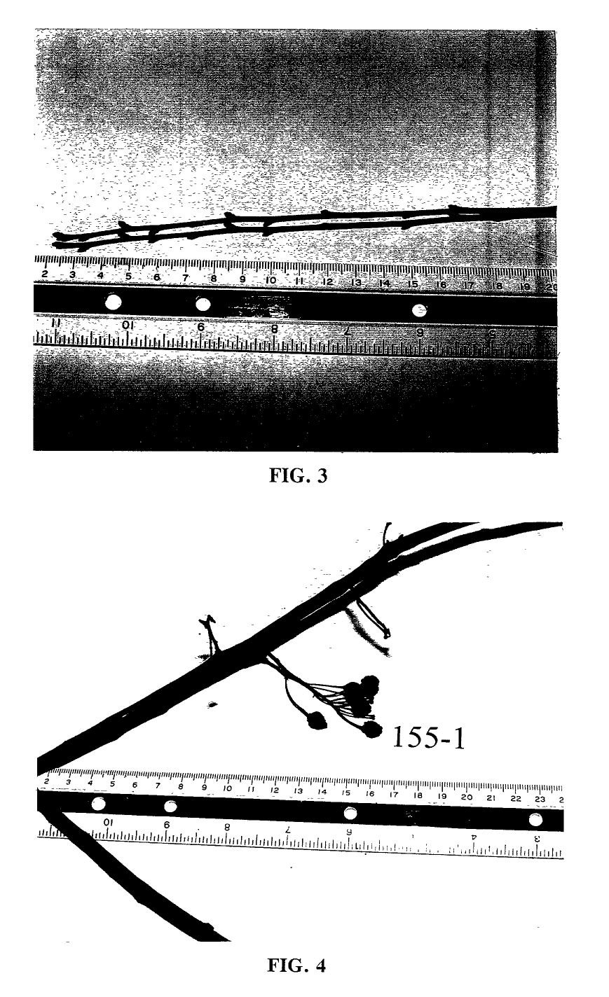

FIG. 3 shows a specimen of typical branch tip with buds of the new cultivar during the winter. Dimensions in centimeters and inches are included.

FIG. 4 shows specimens of typical branches with dried fruit of the new cultivar during the winter. Dimensions in centimeters and inches are included.

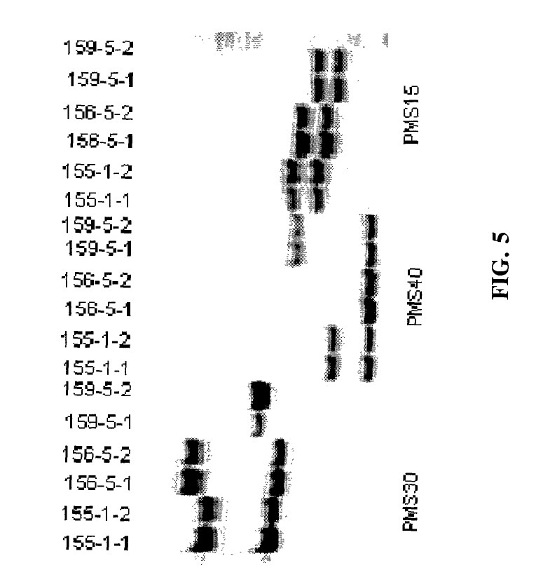

FIG. 5 shows the DNA fingerprint of the new cultivar of the present invention as well as that of the ‘UCMH 56’ and ‘UCMH 59’ cultivars for comparative purposes. Three microsatellite markers were used during the DNA determinations (i.e., PMS30, PMS40 and PMS15). Data with respect to the plants of the new cultivar of the present invention is designated “156-5”. Data with respect to the plants of the ‘UCMH 55’ cultivar is designated “155-1”, and data with respect to the ‘UCMH 59’ cultivar is designated “159-5”.

When preparing the DNA fingerprints, a total of six leaf samples were randomly collected from two different but replicate trees. Accordingly, each genotype was sampled and replicated twice. The two samples are distinguished during the presentation of data by the final digit shown in FIG. 5 (i.e., by “−1” or by “−2”). DNA was extracted using Dneasy Plant Kit from Qiagene, Inc. (Valencia, Calif., U.S.A.) following the manufacturer's protocol. The extracted DNA was purified by adding 1/10 volume 3M sodium acetate and 2 volumes 100 percent ethanol and subsequent storage at −20° C. for an hour. The samples were centrifuged at 13,000 rpm for 15 minutes and the pellets were washed two times with 70 percent ethanol. The pellets were air dried and resolved in 50 μl TE buffer. Quantification of DNA was performed with ethidium bromide agarose gel plates. PCR was carried out under the following conditions: 100-150 ng of template DNA, 250 nM of each primer, 200 μM of dNTPs, 0.5 U of Taq Polymerase, and 1.5 mM of MgCl₂. The reaction was run for 45 cycles (denaturing at 94° C. for 1 minute, annealing at 60° C. for 1 minute, with a two minute extension at 72° C.), followed by a single extension at 72° C. for 60 minutes. The amplification products were detected on 5.5 percent polyacrylamide gels using a Li-Cor IR² 4200 DNA sequencer (Li-Cor, Neb., U.S.A.). The three microsatellite markers clearly distinguished the three cultivars. Both repeats of each rootstock showed identical fingerprints. The marker PMS30 produced two bands for rootstock ‘UCHM 55’ at 132 bp (base pair) and 159 bp, two bands for ‘UCMH 56’ at 132 bp and 168 bp, and one band for ‘UCMH 59’ at 142 bp. Marker PMS40 produced two bands for ‘UCMH 55’ at 92 bp and 111 bp, and one band for ‘UCMH 56’ at 92 bp, and two bands for ‘UCMH 59’ at 92 bp and 129 bp. Marker PMS15 produced two bands for ‘UCMH 55’ at 118 bp and 128 bp, two bands for ‘UCMH 56’ and 112 bp and 123 bp, and two bands for ‘UCMH 59’ at 105 bp and 115 bp.

### DETAILED DESCRIPTION

The following is a detailed description of the new cultivar. The tree was grown at the Experimental Orchards of the University of California at Davis, Calif., U.S.A. Color designations are presented with reference to the “Dictionary of Color” by Maerz and Paul, First Edition (1930).

Botanical Classification: Prunus mahaleb, cv. ‘UCMH 55’.

Tree:

Size.—Intermediate to the ‘UCMH 56’ and ‘UCMH 59’ cultivars.

Growth.—Spreading, the upper canopy is very upright, and two-year-old wood in the lower canopy is pendulous. All wood is very fine. Forms more fine wood than the ‘UCMH 56’ and ‘UCMH 59’ cultivars.

Branches:

Shoot growth form.—Straight with alternate leaves, laterals are highly branched and secondaries are unbranched, and laterals arise at approximately 75 to 90 degrees from the point of origin on the main scaffold.

Size.—Both current and previous season wood is fine and commonly displays a diameter of less than 1 cm.

Spurs.—Commonly 9 to 16 spurs are present per 85 cm of previous season shoot that measure approximately 5 to 7 cm in length.

Internode length.—Approximately 1.5 to 2.5 cm and increasing in length basipetally.

Shoot bark.—Smooth in texture, and the coloration is light brown with a silvery cast varying from Arabian night brown (14 A 11) to 14 G 8.

Lenticels.—Prominent, densely distributed, and under magnification are raised.

Axillary buds.—Borne on spurs of varying length on previous season growth axillary, imbricate, sessile, and single. The bud tips are pointed and the bud pose is adpressed on dormant new wood. The bud support is small.

Leaves:

Bearing.—Simple, alternate, and petioled.

Pose.—Curved outward and downward.

Length.—Approximately 2 to 2.5 cm on the upper canopy and approximately 4 to 5 cm on the lower canopy.

Width.—Approximately 1.5 to 2 cm on the upper canopy and approximately 2.5 to 3.5 cm on the lower canopy.

Form.—Oval on the upper canopy with an acuminate tip and a truncate base, and ovate-elliptic to oval on the lower canopy with an acuminate tip and a truncate base.

Margins.—Crenate and glandular between rounded teeth.

Surfaces.—Glabrous on the dorsal and ventral surfaces with short stiff hairs along the midrib of the ventral surface that are visible with magnification.

Petiole.—Commonly with glands at the leaf-petiole juncture, approximately 1 to 1.5 cm in length on the upper canopy, and approximately 2 to 2.5 cm in length on the lower canopy.

Venation.—Pinnate.

Color.—21 L 6 (Parrot Green) to 21 L 9.

Flowers:

Type.—Single or inflorescent.

Form.—Paniculate with a peduncle of approximately 2 to 2.5 cm in length and a pedicel of approximately 1 to 1.3 cm in length.

Bearing.—On previous season laterals and commonly in clusters of three blossoms.

Color.—White.

Fruit:

Bearing.—Drupe.

Stone shape.—Substantially spherical.

Stone size.—Intermediate and approximately 7 to 8 mm in diameter.

Disease Resistance: The new cultivar has shown 100 percent survival rate in field trials at sites that are heavily infested with Phytophthora spp. and stem pitting virus. At the same sites, approximately 50 percent of the standard Mahaleb plants died.

Vegatative Propagation: The new cultivar asexually reproduces well through the rooting of softwood and hardwood cuttings. The use of softwood cuttings is preferred.

Use as a Scion Rootstock: Field testing has been conducted using a scion of ‘Bing’ cherry following budding. Precocious flowering and cropping are facilitated when using the new cultivar as a rootstock. For instance, flower and fruit production can begin easily in the 4th or 5th growing season with the flowers opening earlier in the season. The fruit size has been found to be comparable to that formed with standard Mahaleb rootstock. To date the new cultivar produced low numbers of root and crown suckers.

I claim:

1. A new and distinct cultivar of Prunus mahaleb plant that exhibits the following combination of characteristics:
(a) readily is amenable to vegetative propagation,
(b) performs well as an understock for cherry production,
(c) forms a tree that is intermediate in size and forms more fine wood when compared to the ‘UCMH 56’ and ‘UCMH 59’ cultivars, and
(d) displays improved resistance to Phytophthora spp.;
substantially as illustrated and described.
